SEQUENCE LISTING (10 sequences)

SEQ ID NO: 1 - DNA, 60 bp, Artificial Sequence
  Primer
tctaatgaagtgaatcgtttagcaacaggacagattccgcgtgtaggctggagctgcttc

SEQ ID NO: 2 - DNA, 60 bp, Artificial Sequence
  Primer
aaaagccaaccgggcggttggctcttcgaaagatttacaccatatgaatatcctccttag

SEQ ID NO: 3 - DNA, 28 bp, Artificial Sequence
  Primer
tatagagctctctgcctgttctgctatg

SEQ ID NO: 4 - DNA, 36 bp, Artificial Sequence
  Primer
ggcgcagatataacgctgcgccgcataagattaggc

SEQ ID NO: 5 - DNA, 36 bp, Artificial Sequence
  Primer
cagcgttatatctgcgccctttcgaagagccaaccg

SEQ ID NO: 6 - DNA, 28 bp, Artificial Sequence
  Primer
tataggtaccgccagttgttcaggtgtg

SEQ ID NO: 7 - DNA, 28 bp, Artificial Sequence
  Primer
tatagagctcgccgctcatgcagattac

SEQ ID NO: 8 - DNA, 36 bp, Artificial Sequence
  Primer
ttacttcaggctgccaaccagcgctttgcggctatc

SEQ ID NO: 9 - DNA, 36 bp, Artificial Sequence
  Primer
gttggcagcctgaagtaatgataatgccccggtcgg

SEQ ID NO: 10 - DNA, 28 bp, Artificial Sequence
  Primer
tataggtaccccaatacgcgtccgcttc

(19) United States
(12) Patent Application Publication
(10) Pub. No.: US 2017/0049872 A1
(43) Pub. Date: Feb. 23, 2017

(54) LIVE SALMONELLA VACCINE AND METHODS TO PREVENT FOWL TYPHOID

(71) Applicant: Arizona Board of Regents on behalf of Arizona State University, Scottsdale, AZ (US)

(72) Inventors: Kenneth Roland, Mesa, AZ (US); Roy Curtiss, Gainesville, FL (US); Pawel Laniewski, Tempe, AZ (US); Arindam Mitra, West Bengal (IN)

(73) Assignee: Arizona Board of Regents on behalf of Arizona State University, Scottsdale, AZ (US)

(21) Appl. No.: 15/308,338

(22) PCT Filed: Apr. 22, 2015
(86) PCT No.: PCT/US15/27148

§ 371 Date: Nov. 1, 2016

Related U.S. Application Data
(60) Provisional application No. 61/987,820, filed on May 2, 2014.

(51) Int. Cl. A61K 39/112

(52) U.S. Cl. CPC A61K 39/0275; A61K 2039/522

(57) ABSTRACT

We constructed S. Gallinarum strains deleted for the global regulatory gene fur (FIG. 1) and evaluated their virulence and protective efficacy in Rhode Island Red chicks and Brown Leghorn layers. The fur deletion mutant was a virulent and, when delivered orally to chicks, elicited excellent protection against lethal S. Gallinarum challenge. We also examined the effect of a pmi mutant and a combination of fur deletions with mutations in the pmi and rfaH genes, which affect O-antigen synthesis, and ansB, whose product inhibits host T cell responses. The ΔAfur Δpmi and Δfur ΔansB double mutants were attenuated, but not protective when delivered orally to chicks. However, a Δpmi Δfur strain was substantially immunogenic when administrated intramuscularly. Altogether our results show that the fur gene is essential for virulence of S. Gallinarum and the fur mutant is effective as a live recombinant vaccine against fowl typhoid.

### CROSS-REFERENCE TO RELATED APPLICATIONS

This application claims priority to U.S. Provisional Patent Application No. 61/987,820 filed on May 2, 2014.

### STATEMENT OF FEDERALLY SPONSORED RESEARCH OF DEVELOPMENT

This invention was made with government support under 0965511 awarded by The National Science Foundation. The government has certain rights in the invention.

### BACKGROUND OF THE INVENTION

S. Gallinarum causes fowl typhoid, recognized worldwide as a socially and economically important disease. It is a septicemic disease mainly affecting chickens and turkeys, although natural infections in a number of wild birds, including ducks, pheasants ostriches, peacocks and quail have been reported. Fowl typhoid has been essentially eradicated in many developed countries while it remains an important economic problem in many other areas of the world, including Tanzania, Zambia, Libya, Nigeria, and Morocco.

In Tanzania for example, it is the most important disease affecting commercial chickens, where it is a particularly important pathogen for commercial layers. Fowl typhoid is a significant economic problem in Mexico and Central and South America. In the countries listed above, many of which have a high ambient temperature, the possibilities of disease prevention by a combination of hygiene and housing improvements are limited.

There remains nothing in current data indicating the magnitude or economic consequences of S. Gallinarum infection. Currently, there is only one live vaccine in use for the prevention of fowl typhoid. In the 1950s, a rough (lacking the complete lipopolysaccharide (LPS) O-antigen) live vaccine, S. Gallinarum strain 9R, was developed. LPS is a unique lipid found in the outer membrane of all gram negative bacteria, such as Salmonella. LPS is composed of a hydrophobic domain called lipid A, a nonrepeating “core” oligosaccharide and a distal polysaccharide called O-antigen. While this vaccine is protective, there are several drawbacks.

First, the 9R vaccine possesses an uncharacterized attenuation lesion(s) and, despite its apparent safety, the risk of reversion to wildtype is unknown. The second drawback is that it is administered subcutaneously to birds at 6 weeks of age, followed by two booster inoculations at 14 and 16 weeks of age, meaning that every immunized bird must be physically handled three times and materials for injection (needles, syringes, etc) must be available, adding to the expense of vaccination.

Therefore, improvements in a vaccine for fowl typhoid and methods for providing a new fowl typhoid vaccine are desirable.

### SUMMARY OF THE INVENTION

The disclosure herein relates to live Salmonella Gallinarum vaccine with defined mutations that can be applied orally or by intramuscular injection to poultry for the prevention of fowl typhoid.

While Salmonella fur deletions have been noted as being attenuated, to varying degrees, the general consensus of previous work is that they are not immunogenic in healthy animals. This work represents the first time that a fur mutant has been shown to be both highly attenuated and substantially immunogenic.

In an embodiment, a strain of S. Gallinarum bacteria includes a mutation that prohibits the strain from synthesizing a functional ferric uptake regulator protein and wherein the strain is attenuated and immunogenic in fowl.

In an embodiment, a strain of S. Gallinarum bacteria includes a mutation that prohibits the strain from synthesizing phosphomannose isomerase and wherein the strain is attenuated and immunogenic in fowl.

In a further embodiment, a vaccine for inoculation against S. Gallinarum infection includes a pharmaceutically acceptable carrier and a strain of S. Gallinarum that further includes a mutation that prohibits the strain from synthesizing a functional ferric uptake regulator protein.

In another embodiment, a method of making a vaccine includes providing a strain of S. Gallinarum including a mutation that prohibits the strain from synthesizing a functional ferric uptake regulator protein, wherein the strain is attenuated and immunogenic and incorporating the strain into a pharmaceutically acceptable carrier.

In another embodiment, a method of vaccinating fowl against typhoid includes the administration of a strain of S. Gallinarum, which includes a mutation that prohibits the strain from synthesizing a functional ferric uptake regulator protein, in a pharmaceutically acceptable carrier.

In yet another embodiment, a method of vaccinating fowl against typhoid includes the administration of a strain of S. Gallinarum, which includes a mutation that prohibits the strain from synthesizing phosphomannose isomerase, in a pharmaceutically acceptable carrier.

These and other aspects of the invention will be apparent upon reference to the following detailed description and figures. To that end, any patent and other documents cited herein are hereby incorporated by reference in their entirety.

### BRIEF DESCRIPTION OF THE DRAWINGS

FIG. 1 illustrates phenotype characterization of S. Gallinarum Δfur mutants. A. Fur production in S. Gallinarum wild-type (287/91), Δfur-453::cam (χ11575) and Δfur-712 vaccine strains (χ11798, χ11820, χ11821, χ11823). Whole-cell lysates were obtained from overnight cultures, electrophoresed on a 12% SDS-PAGE gel, transferred onto nitrocellulose and probed with anti-Fur serum. The blot was also probed with anti-GroEL antibodies to serve as a loading control. B. IROMPs production in S. Gallinarum vaccine strains. OMPs were obtained by Sarkosyl-extraction from overnight cultures, electrophoresed on a 10% SDS-PAGE gel and stained with Coomassie blue. C. Effect of acid shock on viability of S. Gallinarum fur mutants. Strains 287/91 (wt), χ11741 (Δpmi-2426), χ11797 (Δfur-712) and χ11798 (Δpmi-2426 Δfur-712) were grown in LB to early logarithmic phase, washed in E medium (pH 7.0) and then challenged with E medium (pH 3.0). Survival was monitored by plating samples on LB agar. The data shown are means and SEM from four independent experiments. A statistical analysis was carried out using two-way ANOVA followed by Tukey's multiple comparison test. All possible pairs of data within each time point except 287/91 vs. χ11741 were significantly different (P<0.01).

FIG. 2 Illustrates a Colonization of spleen and liver in birds that survived the challenge with wild-type S. Gallinarum. Survivors from Expt. 3 (Table 2) were euthanized nineteen days post infection and spleens and livers were collected to recover viable S. Gallinarum from each tissue. Organs were homogenized, diluted and plated on LB agar. Negative samples were additionally enriched using RV broth and plated on SS agar.

### DETAILED DESCRIPTION OF THE INVENTION

Salmonella enterica serovar Gallinarum biovar Gallinarum (S. Gallinarum) is a host-adapted pathogen that causes fowl typhoid—an important disease of poultry. Fowl typhoid is a septicemic disease with a typically short course and significant morbidity and mortality, which can reach as high as 100%. The disease occurs primarily in mature flocks, although birds of all ages may be infected. Resistance to S. Gallinarum also varies with the species and breed. Among chickens, heavier breeds such as Rhode Island Red are more susceptible than lighter breeds such as white leghorns. Fowl typhoid has been eradicated from commercial poultry in many developed countries including the USA and Canada through isolation and removal of contaminated flocks and implementing biosecurity and hygiene management.

Nevertheless, it still constitutes a considerable economic problem for poultry growers, both small backyard farmers and larger commercial operations in many parts of the world such as Central and South America, Africa and Asia, where control measures are insufficient and the climate favors spread of S. Gallinarum in the environment.

We introduce a live attenuated S. Gallinarum vaccine for fowl typhoid that can be applied orally, by coarse spray or by injection in one or two doses. Fowl typhoid is a devastating disease of poultry caused by Salmonella enterica serovar Gallinarum. This disease is currently controlled in the developed world by culling of diseased flocks, good husbandry practices and vaccination. However, in the developing world, this disease is still rampant and accounts for economic losses ranging from 10% of all poultry death due to disease and reduced egg output in survivors. The current vaccine for fowl typhoid is an injectable live Salmonella Gallinarum vaccine that requires 3 doses.

Fur (ferric uptake regulator) acts as a repressor of many genes whose products are involved in iron, zinc and manganese acquisition and uptake. One notable class of Fur-regulated proteins is the iron regulated outer membrane proteins (IROMPs), which serve as receptors for iron siderophore complexes. The genes for these proteins are repressed by Fur when iron is abundant and are up regulated when iron is limiting. Animal hosts restrict iron from invading bacteria during infection, a phenomenon known as “nutritional immunity”. Thus, mechanisms for iron acquisition are important to the pathogenicity of many microorganisms including Salmonella sp.

Fur can also act as a transcriptional activator by enhancing RNAP recruitment, regulating production of small RNAs or functioning as an antirepressor. In Salmonella, Fur also modulates expression of genes involved in acid shock and adaptation and oxidative stress resistance. It further plays a role in regulation of the Salmonella pathogenicity island 1 (SPI1) genes (e.g. hilA and hilD) necessary for invasion. S. Typhimurium strains with an arabinoseregulated fur genotype (fur expressed in vitro in presence of arabinose, not expressed in vivo where arabinose is not available) were partially attenuated and substantially immunogenic in mice. The same study also showed that the attenuation of S. Typhimurium arabinose regulated fur mutants is correlated with the level of the Fur expression.

Furthermore, an S. Enteritidis Δfur strain was partially attenuated and immunization of mice with this strain resulted in decrease of bacterial load in systemic organs after challenge with the wildtype strain. A fur deletion was also employed to improve the safety of a S. Typhimurium ΔssaV mutant. The ΔssaV Δfur double mutant was safe and immunogenic in immunocompromised mice. The pmi gene encodes phosphomannose isomerase that facilitates the interconversion of fructose6phosphate into mannose6phosphate, which is subsequently converted into GDPmannose—a substrate for incorporation into LPS O-antigen side chains. Thus Δpmi mutants cannot produce O-antigen unless an exogenous source of mannose is present.

In the context of a vaccine, Δpmi strains are grown in vitro in the presence of mannose and synthesize a complete O-antigen, a requirement for optimal host colonization. The O-antigen is subsequently lost after several generations of growth in animal tissues, which are devoid of free nonphosphorylated mannose. S. Typhimurium pmi mutants are substantially immunogenic and partially attenuated in mice.

The vaccine described herein is comprised of S. Gallinarum strain(s) with deletions in the global regulatory gene fur and/or pmi. The fur mutant is fully attenuated and protective when administered orally or by injection. This was demonstrated in two chicken breeds: Rhode Island Red and brown leghorn. A fur pmi double mutant is protective when administered by intramuscular injection into brown leghorns.

Vaccination of chickens seems to be the most effective strategy to control fowl typhoid in developing countries where S. Gallinarum is endemic. The rough S. Gallinarum 9R strain is the most widely used vaccine. While somewhat effective, a number of drawbacks have been noted: variability in protective efficacy between breeds; persistence in immunized chickens leading to transmission through eggs and residual virulence in some breeds. Moreover, the means of attenuation is not well defined genetically. Until recently, the attenuation of this strain was believed to be due solely to a defect in lipopolysaccharide (LPS) synthesis. However, recent comparative analysis of its proteome and transcriptome has showed that 9R may also be impaired for the regulation of several virulence factors.

In our efforts to develop safe and efficacious fowl typhoid vaccine candidates we have been examining mutations in global virulence regulators and genes that affect O-antigen synthesis, with emphasis on genes required for virulence in S. Typhimurium. For example, modification or deletion of the global regulator gene crp in S. Gallinarum results in a strain that is safe and efficacious against challenge with virulent S. Gallinarum.

Conversely, mutations in rfc (wzy), required for complete O-antigen synthesis, are attenuating in S. Typhimurium, but have no affect on the virulence of S. Gallinarum when delivered by the oral route. In addition, an arabinose-regulated rfaH construction that results in arabinose-regulated O-antigen synthesis was partially attenuating in S. Typhimurium, but was not attenuating in S. Gallinarum. These results demonstrate that it is not possible to make accurate predictions regarding the virulence and immunogenicity of S. Gallinarum mutants in chickens based on S. Typhimurium mutant data in mice. We decided to expand our approach and explore additional genes involved in global regulation or O-antigen synthesis.

Fur (ferric uptake regulator) acts as a repressor of many genes whose products are involved in iron, zinc and manganese acquisition and uptake. One notable class of Fur-regulated proteins is the iron-regulated outer membrane proteins (IROMPs), which serve as receptors for iron-siderophore complexes. The genes for these proteins are repressed by Fur when iron is abundant and are up regulated when iron is limiting. Animal hosts restrict iron from invading bacteria during infection, a phenomenon known as “nutritional immunity”. Thus, mechanisms for iron acquisition are crucial to the pathogenicity of many microorganisms including Salmonella sp. Fur can also act as a transcriptional activator by enhancing RNAP recruitment, regulating production of small RNAs or functioning as an antirepressor. In Salmonella, Fur also modulates expression of genes involved in acid shock and adaptation and oxidative stress resistance and it plays a role in regulation of the Salmonella pathogenicity island 1 (SPI-1) genes (e.g. hilA and hilD) necessary for invasion.

S. Typhimurium strains with an arabinose-regulated fur genotype (fur expressed in vitro in presence of arabinose, not expressed in vivo where arabinose is not available) were partially attenuated and substantially immunogenic in mice. The same study also showed that the attenuation of S. Typhimurium arabinose-regulated fur mutants is correlated with the level of the fur expression. Furthermore, an S. Enteritidis Δfur strain was attenuated and immunization of mice with this strain resulted in decrease of bacterial load in systemic organs after challenge with the wild-type strain. A fur deletion was also employed to improve the safety of a S. Typhimurium ΔssaV mutant. The ΔssaV Δfur double mutant was safe and immunogenic in immunocompromised mice.

The pmi gene encodes phosphomannose isomerase that facilitates the interconversion of fructose-6-phosphate into mannose-6-phosphate, which is subsequently converted into GDP-mannose—a substrate for incorporation into LPS O-antigen side chains. Thus Δpmi mutants cannot produce O-antigen unless an exogenous source of mannose is present. In the context of a vaccine, Δpmi strains are grown in vitro in the presence of mannose and synthesize a complete O-antigen, a requirement for optimal host colonization. The O-antigen is subsequently lost after several generations of growth in animal tissues, which are devoid of free non-phosphorylated mannose. S. Typhimurium pmi mutants are substantially immunogenic and partially attenuated in mice.

The primary focus of this work is to evaluate the virulence and immunogenicity of S. Gallinarum strains with deletions in the global regulatory gene fur and/or in pmi. We also examined the impact of the fur deletion in combination with several other mutations. Strains were screened for virulence and protective efficacy in two chicken breeds: Rhode Island Red and brown leghorn, which are differently susceptible to fowl typhoid. Our results showed that immunization with an S. Gallinarum fur mutant provided excellent protection against challenge with virulent S. Gallinarum in both breeds.

### Materials and Methods

Bacterial Strains, Plasmids, Media and Growth Conditions.

Bacterial strains and plasmids used in this study are listed in Table 1.

TABLE 1
Bacterial strains and plasmids used in this study.

| | | |
|---|---|---|
| Name | Relevant characteristics | Source |

E. coli strains

| | |
|---|---|
| χ7213 | thi-1 thr-1 leuB6 glnV44 fhuA21 lacY1 recA1 |
| | RP4-2-Tc::Mu [λpir] ΔasdA4 Δ(zhf-2::Tn10); used |
| | for conjugational transfer of suicide plasmids |
| χ7232 | endA1 hsdR17 (r_{K}⁻ m_{K}⁺) gln V44 thi-1 recA1 gyrA |
| | relA1 Δ(lacZYA-argF)U169 λpir deoR (φ80dlac |
| | Δ(lacZ)M15); used for general cloning |

S. Gallinarum strains

| | | |
|---|---|---|
| χ4173 | wild-type challenge strain | |
| 287/91 | wild-type vaccine parent strain | |
| χ11575 | Δfur-453::cam | 287/91 |
| χ11386 | ΔP_{rfaH178}::TT araC P_{BAD} rfaH | 287/91 |
| χ11741 | Δpmi-2426 | 287/91 |
| χ11797 | Δfur-712 | 287/91 |
| χ11798 | Δpmi-2426 Δfur-712 | χ11741 |
| χ11820 | Δfur-712 Δpmi-2426 | χ11797 |
| χ11821 | ΔP_{rfaH178}::TT araC P_{BAD} rfaH Δfur-712 | χ11386 |
| χ11822 | ΔansB1235 | 287/91 |
| χ11823 | Δfur-712 ΔansB1235 | χ11797 |

Plasmids

| | | |
|---|---|---|
| pKD46 | λ red expression vector | |
| pRE112 | sacB mobRP4; R6K ori; Cm^{R} | |
| pYA3546 | suicide vector for introduction Δpmi-2426 | |
| pYA5239 | suicide vector for introduction Δfur-712 | pRE112 |
| pYA5272 | suicide vector for introduction ΔansB1235 | pRE112 |

### Working Example 1

Escherichia coli and S. Gallinarum strains were routinely cultured at 37° C. in LB broth or on LB agar. Cultures of S. Gallinarum mutants were supplemented with 0.05% mannose (Sigma-Aldrich, St. Louis, Mo.) (for Δpmi-2426), 0.2% arabinose (Sigma-Aldrich) (for ΔPrfaH178::TT araC PBAD rfaH, hereafter ΔPrfaH178) or chloramphenicol (15 μg/ml; Sigma-Aldrich) (for Δfur-453::cam). Carbohydrate-free nutrient broth (NB) was used for growth when determining LPS profiles. Strains were grown in NB without mannose (for pmi strains) or arabinose (for ΔPrfaH178 strains) overnight and subcultured (1:100) into fresh NB with or without the appropriate sugar for a second passage. LB agar without sodium chloride and with 7.5% sucrose (Sigma-Aldrich) was employed for sacB-based counterselection. MacConkey plates with 1% mannose were used to indicate sugar fermentation.

For animal experiments, S. Gallinarum strains were cultured in LB broth with appropriate supplements. Overnight cultures were diluted 1:100 and grown with shaking (200 rpm) to an optical density at 600 nm of 0.8. Then, bacteria were centrifuged at 5,000×g for 15 min at room temperature and resuspended in phosphate-buffered saline (PBS) or buffered saline with 0.01% gelatin (BSG). LB or Salmonella Shigella (SS) agar plates were used to enumerate S. Gallinarum recovered from chicken tissues. Rappaport-Vassiliadis R10 (RV) broth was employed to enrich samples for S. Gallinarum. All media were purchased from BD Difco (Franklin Lakes, N.J.) unless otherwise indicated.

General DNA procedures. DNA manipulations, including plasmid and genomic DNA isolation, restriction enzyme digestions, ligations and other DNA-modifying reactions, were carried out as described by Sambrook and Russell or were performed according to the manufacturers' instructions (New England Biolabs, Ipswich, Mass.; Qiagen, Valencia, Calif.; Promega, Madison, Wis.). Synthesis of primers (Table 2) and DNA sequencing were performed by Integrated DNA Technologies (Coralville, Iowa) and DNA Laboratory at Arizona State University (Tempe, Ariz.), respectively. Polymerase chain reactions (PCR) were carried out with Klentaq LA polymerase (DNA Polymerase Technology, St. Louis, Mo.), possessing proofreading activity. Recombinant plasmids were introduced into E. coli and S. Gallinarum cells by transformation or electroporation, respectively.

Construction of S. Gallinarum vaccine strains. All vaccine candidates were derived from S. Gallinarum strain 287/91. The fur deletion/insertion mutation Δfur-453::cam was constructed via the λ red recombination method. Flanking sequences were based on the S. Gallinarum 287/91 genome using primers AM-115 and AM-116 (Table 2).

TABLE 2
Primers used in this study.

| | | | Restriction |
|---|---|---|---|
| Name | Sequence (5′→3′) | Orientation | site |
| AM-115 | TCTAATGAAGTGAATCGTTTAGCA | forward | |
| | ACAGGACAGATTCCGCGTGTAGGCT | | |
| | GGAGCTGCTTC (SEQ ID NO. 1) | | |
| AM-116 | AAAAGCCAACCGGGCGGTTGGCTC | reverse | |
| | TTCGAAAGATTTACACCATATGAAT | | |
| | ATCCTCCTTAG (SEQ ID NO. 2) | | |
| fur-1F | TATAGAGCTCTCTGCCTGTTCTGCT | forward | SacI |
| | ATG (SEQ ID NO. 3) | | |
| fur-1R | GGCGCAGATATAACGCTGCGCCGC | reverse | |
| | ATAAGATTAGGC (SEQ ID NO. 4) | | |
| fur-2F | CAGCGTTATATCTGCGCCCTTTCGAA | forward | |
| | GAGCCAACCG (SEQ ID NO. 5) | | |
| fur-2R | TATAGGTACCGCCAGTTGTTCAGGT | reverse | KpnI |
| | GTG (SEQ ID NO. 6) | | |
| ansB-1F | TATAGAGCTCGCCGCTCATGCAGAT | forward | SacI |
| | TAC (SEQ ID NO. 7) | | |
| ansB-1R | TTACTTCAGGCTGCCAACCAGCGC | reverse | |
| | TTTGCGGCTATC (SEQ ID NO. 8) | | |
| ansB-2F | GTTGGCAGCCTGAAGTAATGATAAT | forward | |
| | GCCCCGGTCGG (SEQ ID NO. 9) | | |
| ansB-2R | TATAGGTACCCCAATACGCGTCCGC | reverse | KpnI |
| | TTC (SEQ ID NO. 10) | | |

Nucleotides underlined denote restriction enzyme sites used for cloning. Nucleotides bolded are complementary to the S. Gallinarum 287/91 chromosome.

All other gene replacements were introduced by conjugational transfer of suicide plasmids using donor E. coli strain χ7213.

To construct the Δfur-712 deletion, fur flanking regions were amplified from the S. Gallinarum 287/91 genome by two-step PCR. Firstly, 644 bp and 663 bp DNA fragments flanking fur gene were amplified with fur-1F/-1R and fur-2F/-2R primer sets (Table 2), respectively. Thereafter; the mix of PCR products was used as a template in the next amplification reaction with fur-1F and fur-2R primers. The 1.3 kb DNA fragment was digested with SacI/KpnI restriction enzymes and cloned into suicide plasmid vector pRE112. The resulting suicide plasmid, pYA5239, carried a deletion of the entire fur gene including 251 bp promoter region. The Δfur-712 mutation was introduced by allelic exchange into S. Gallinarum strains 287/91, χ11741 and χ11386 to generate χ11797 (Δfur-712), χ11798 (Δpmi-2426 Δfur-712) and χ11821 (ΔPrfaH178::TT araC PBAD rfaH Δfur-712), respectively.

The ΔansB1235 deletion was constructed as described above using ansB-1F/-1R and ansB-2F/-2R primer pairs (Table 2). The resulting suicide plasmid, pYA5272, carried a deletion of the entire ansB gene including the 188 bp promoter sequence. The ΔansB1235 mutation was introduced into S. Gallinarum strains 287/91 and χ11797 to generate χ11822 (ΔansB1235) and χ11823 (Δfur-712 ΔansB1235), respectively.

As S. Typhimurium and S. Gallinarum share >99% sequence similarity in the flanking region surrounding pmi, previously constructed suicide plasmid pYA3546 carrying S. Typhimurium DNA sequences was used to create S. Gallinarum Δpmi-2426 mutants (25, 32). Plasmid pYA3546 was introduced by conjugation into S. Gallinarum strains 287/91 and χ11797 to generate χ11741 (Δpmi-2426) and χ11820 (Δfur-712 Δpmi-2426), respectively.

All mutations were verified by PCR. We confirmed Fur production, or lack thereof, by western blotting. The Δpmi mutation was confirmed by white colony phenotype on mannose-MacConkey agar. LPS profiles were examined by silver staining in 12% polyacrylamide gels as described previously.

Isolation of outer membrane proteins (OMPs). OMPs were isolated using the Sarkosyl-extraction method.

SDS-PAGE and western blotting. SDS-PAGE and western blotting procedures were done by standard techniques. Blots were developed with nitro blue tetrazolium chloride/5-bromo-4-chloro-3′-indolyl phosphate (Amresco, Solon, Ohio) as a substrate, using rabbit polyclonal anti-Fur serum or anti-GroEL antibodies (Sigma-Aldrich) as primary antibodies and mouse anti-rabbit IgG alkaline phosphatase conjugate (Sigma-Aldrich) as secondary antibodies.

Acid shock assay. Acid resistance was evaluated essentially as previously described, with a few modifications. Strains were grown aerobically in LB broth with appropriate supplements until they reached an optical density of ˜0.4. Culture aliquots were centrifuged (10 min 5000×g) at room temperature and bacterial pellets were washed with E medium (pH 7.0). Thereafter, cells were centrifuged again and resuspended at a density of ˜0.5×109 CFU/ml in E medium (pH 3.0). Acid challenge was conducted at 37° C., and samples were collected immediately after resuspension and in 30-minute intervals. Samples were serially diluted and plated onto LB agar to assess bacterial viability.

Animal supply and housing. Animal experiments were performed using two breeds of chickens: Rhode Island Reds and Brown Leghorns. Straight run Rhode Island Red chicks were obtained from Randall Burkey Co. (Boerne, Tex.) or Murray McMurray Hatchery (Webster City, Iowa) one or two days after hatch. Birds were housed in separate cages for each group and given water and feed ad libitum. All animal experiments were carried out in compliance with Institutional Animal Care and Use Committee (IACUC) and Animal Welfare Act at Arizona State University.

Female Brown Leghorn chickens were hatched in-house. Chickens were feed Purina Lab Chow 5065, water and feed available ad libitum. Six-week old chickens were distributed among several isolators and tagged.

Determination of lethal dose, 50% (LD50). Strains were grown and harvested as described above in section “Bacterial strains, plasmids, media and growth conditions”. Bacterial pellets were resuspended in PBS or BSG and adjusted to achieve a dose of ˜102 to ˜108 CFU in a volume of 100 μl for orally inoculating chicks. The virulence of wild-type strain, 287/91, and its derivatives were assessed in three- or five-day-old Rhode Island Red chicks. Birds were observed for fowl typhoid symptoms for three weeks post inoculation. Deaths were recorded daily. The LD50 was calculated using the Reed and Muench method.

Immunization and challenge regimen. For Rhode Island Reds, three- or five-day-old chicks were inoculated orally with 100 μl of PBS containing ˜1×108 CFU of the appropriate S. Gallinarum strain and boosted with the same strain and dose two weeks later. No food was provided for ˜5-6 h prior to immunizations or challenge. Groups of birds inoculated with buffer (PBS or BSG) served as controls. At four weeks of age (i.e. two weeks post booster), all birds were orally challenged with ˜1×107 CFU of heterologous S. Gallinarum strain χ4173. Note that in the case of fur::cam deletion/insertion strain χ11575, all chicks survived the virulence study described above. They were then treated as vaccinated chicks, boosted with ˜1×108 CFU of χ11575 and challenged.

Chickens were observed for fowl typhoid symptoms for 3 weeks post challenge. Deaths were recorded daily. At the end of the observation period, surviving birds were euthanized and their organs were inspected for lesions. Spleens and livers were collected and homogenized. Dilutions of the homogenate were made in BSG and plated onto LB agar plates for enumeration of Salmonella present in each tissue. Enrichment with RV broth and subsequent plating onto SS agar plates was carried out for organ samples in which no Salmonella was detected by direct plating.

For experiments with Brown Leghorns, groups of 15 or 16 seven-week-old pullets were immunized orally (˜2×107−2×108 CFU in most cases) or intramuscularly (˜2×104 or ˜2×107 CFU) of the appropriate S. Gallinarum strain. A group of non-vaccinated birds was used as a control. At ten weeks of age (i.e. three weeks post immunization) all birds were orally challenged with ˜2×108 CFU of homologous S. Gallinarum strain 287/91. Birds were monitored for 3 weeks post-challenge. Then, surviving birds were euthanized and necropsies performed to determine the presence of tissue lesions.

Statistical analyses. All statistical analyses were performed using GraphPad Prism 6 (GraphPad Software, San Diego, Calif.). The significance of differences between the obtained values was appraised using two-way analysis of variance (ANOVA) followed by Tukey's or Dunnett's tests. P values <0.05 were considered significant.

### Results

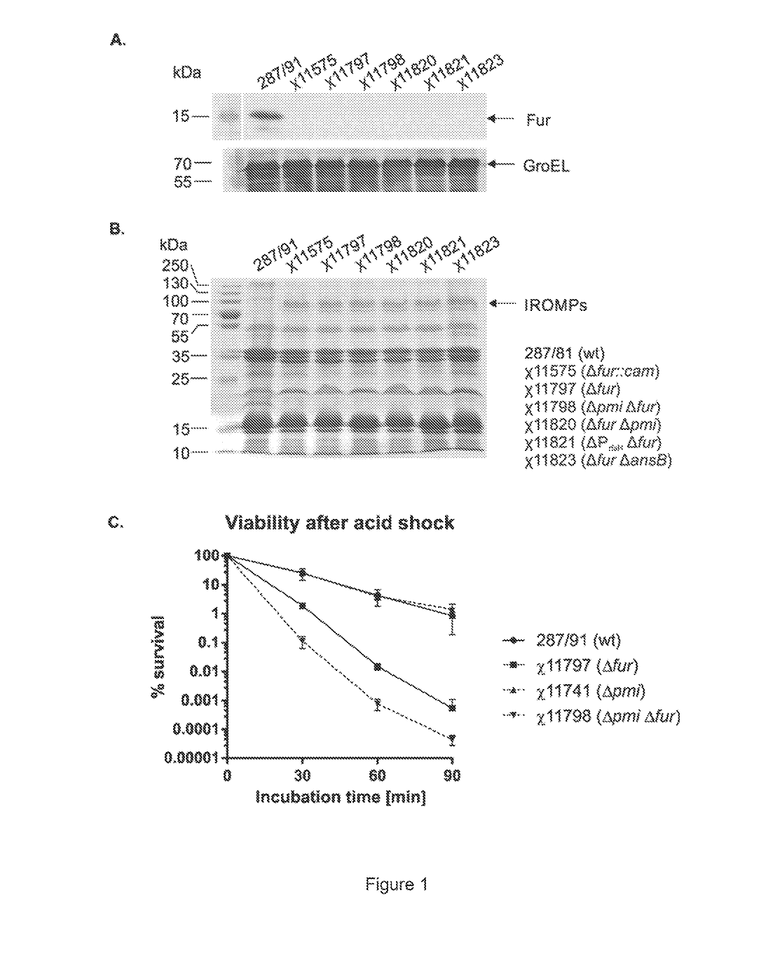

Screening for S. Gallinarum immunogenic mutants. To evaluate the impact of a fur deletion in S. Gallinarum, we constructed strain χ11575, harboring the Δfur-453::cam deletion/insertion. As expected, Fur was not detected in χ11575 by western blot analysis (FIG. 1A). Then we screened for production of IROMPs after growth in LB, a medium in which iron is not limiting (˜7.6 μM iron). Under these conditions, IROMPs were not detected in parent strain 287/91, but were easily detectable in χ11575 (FIG. 1B). The three distinct bands with approximate molecular masses of 83, 78 and 74 kDa correspond to the predicted molecular masses of the Fur-regulated IROMPs FepA, IroN and Cir, respectively (FIG. 1B). The protein pattern is in an agreement with previous observations of S. Typhimurium outer membrane preparations from wild-type cells grown under iron-limiting conditions or from a fur mutant grown in the relatively iron-rich medium, NB.

Strain χ11575 was then screened for virulence in Rhode Island Red chicks. Birds were given orally graded doses of bacteria and monitored for three weeks. The strain was fully attenuated with no deaths occurring at the highest dose tested (LD50>˜1×108 CFU) (Table 3).

TABLE 3
Attenuation of S. Gallinarum mutants in Rhode Island Red chickens.

| Strain | Genotype | LD₅₀ (CFU) |
|---|---|---|
| 287/91 | wild type | 6.7 × 10⁴ |
| χ11575 | Δfur-453::cam | >~1 × 10⁸ |
| χ11797 | Δfur-712 | >0.9 × 10⁸ |
| χ11741 | Δpmi-2426 | 1.0 × 10⁷ |
| χ11822 | ΔansB1235 | 1.3 × 10⁴ |
| χ11821 | ΔP_{rfaH178} Δfur-712 | >1.2 × 10⁸ |

Encouraged by these results, we evaluated the ability of χ11575 to confer protection against challenge with virulent S. Gallinarum. The same chicks used in the virulence assay were boosted two weeks after the first inoculation with ˜1×108 CFU of χ11575 and challenged two weeks later with ˜1×107 CFU of heterologous wild-type strain χ4173. All the birds, even those primed with the lowest dose (˜1×102 CFU) of χ11575, survived challenge with virulent S. Gallinarum strain (Table 4), suggesting that an S. Gallinarum fur mutant is a viable vaccine candidate. However, strain χ11575 contains a chloramphenicol resistance cassette in the chromosome, precluding its use as a vaccine. Thus, we constructed S. Gallinarum strain χ11797, carrying the unmarked Δfur-712 deletion (Table 1). We confirmed the absence of detectable Fur in this strain (FIG. 1A) and production of IROMPs following growth in LB was indistinguishable from χ11575 (FIG. 1B).

In S. Typhimurium, fur mutants display an acid-sensitive phenotype. To determine acid resistance of S. Gallinarum fur mutants, χ11797 (Δfur-712) and parent strain 287/91 were cultured in LB to early logarithmic phase of growth and then challenged at pH 3.0. The percentage of viable cells during low-pH challenge declined more rapidly for χ11797 than for 287/91 (FIG. 1C). After 30 min of pH 3.0 exposure, survival of the mutant was significantly lower (2.0%) compared to that of the wild type (25.1%; P<0.01). After 90 min of challenge only 0.001% of χ11797 cells survived compared to 0.847% of the wild type, corresponding to a ˜880-fold (2.9 log) difference in the number of viable cells (P<0.0001).

Virulence and protective efficacy of S. Gallinarum Δfur-712 mutant in Rhode Island Red chickens. We determined the virulence of S. Gallinarum χ11797 (Δfur-712) in five-day-old Rhode Island Red chicks. As expected, strain χ11797 was fully attenuated (LD50>0.9×108 CFU) (Table 3). In contrast, parent strain 287/91 was highly virulent, with an LD50 of 6.7×104 CFU, consistent with previous results.

Strain χ11797 was then evaluated for its ability to induce protective immunity against challenge with a virulent S. Gallinarum strain. Two independent protection experiments were performed on five-day-old Rhode Island Red chickens. Birds were primed and boosted orally two weeks later with identical doses of ˜1×108 CFU of χ11797. In each study a control group was given a sterile buffer instead of vaccine. At four weeks of age, all birds were challenged with ˜1×107 CFU of heterologous virulent strain χ4173. In both studies immunization with strain χ11797 provided significant protection compared to non-immunized control birds (Table 4).

TABLE 4
Protective efficacy of S. Gallinarum fur mutants in Rhode Island Red chickens^{a}.

| | | | | | | | | | |
|---|---|---|---|---|---|---|---|---|---|
| | | | | | | | Heart | | |
| | | | Prime | Boost | Hepato- | Spleno- | lesions/ | Alive/ | Percent |
| Strain | Genotype | Exp | (CFU) | (CFU) | megaly | megaly | pericarditis | total | survival |

Single fur

| | | | | | | | | | |
|---|---|---|---|---|---|---|---|---|---|
| χ11575 | Δfur- | 1 | Range of | ~1 × 10⁸ | NT^{c} | NT | NT | 20/20 | 100%^{d} |
| | 453::cam | | doses^{b} | | | | | | |
| χ11797 | Δfur-712 | 2 | 1.0 × 10⁸ | 1.2 × 10⁸ | NT | NT | NT | 10/11 | 91%^{d} |
| | | 3 | 1.2 × 10⁸ | 1.1 × 10⁸ | 1/13 (8%)^{d} | 2/13 (15%)^{d} | 5/13 (38%) | 12/13 | 92%^{d} |

fur combined with other mutations

| | | | | | | | | | |
|---|---|---|---|---|---|---|---|---|---|
| χ11798 | Δpmi- | 2 | 1.0 × 10⁸ | 1.0 × 10⁸ | NT | NT | NT | 2/9 | 22% |
| | 2426 | | | | | | | | |
| | Δfur-712 | | | | | | | | |
| χ11820 | Δfur-712 | 3 | 1.2 × 10⁸ | 1.3 × 10⁸ | 10/12 (83%) | 9/12 (75%) | 3/12 (25%) | 4/12 | 33% |
| | Δpmi- | | | | | | | | |
| | 2426 | | | | | | | | |
| χ11821 | ΔP_{rfaH178} | 3 | 1.1 × 10⁸ | 1.1 × 10⁸ | 9/12 (75%) | 8/12 (67%) | 3/12 (25%) | 4/12 | 33% |
| | Δfur-712 | | | | | | | | |
| χ11823 | Δfur-712 | 3 | 1.0 × 10⁸ | 0.9 × 10⁸ | 7/12 (58%) | 8/12 (67%) | 3/12 (25%) | 6/12 | 50% |
| | ΔansB1235 | | | | | | | | |

Controls

| | | | | | | | | | |
|---|---|---|---|---|---|---|---|---|---|
| BSG | — | 1 | — | — | NT | NT | NT | 2/20 | 10% |
| PBS | — | 2 | — | — | NT | NT | NT | 2/11 | 18% |
| PBS | — | 3 | — | — | 10/12 (83%) | 10/12 (83%) | 1/12 (8%) | 2/12 | 17% |

^{a}Three- or five-day-old chicks were immunized orally with the indicated dose of S. Gallinarum and boosted two weeks later. At four weeks of age all birds were challenged with ~1 × 10⁷ CFU of heterologous S. Gallinarum wild-type strain (χ4173).
^{b}These birds were survivors of the virulence assay so the chicks received 1 × 10², 10⁴, 10⁶ or 10⁸ CFU as a priming dose. The boost was 1 × 10⁸ CFU for all birds.
^{c}not tested
^{d}P < 0.01 compared to control

In both experiments, >90% of the vaccinated chickens survived compared to only 17-18% survival in the control groups (P<0.001).

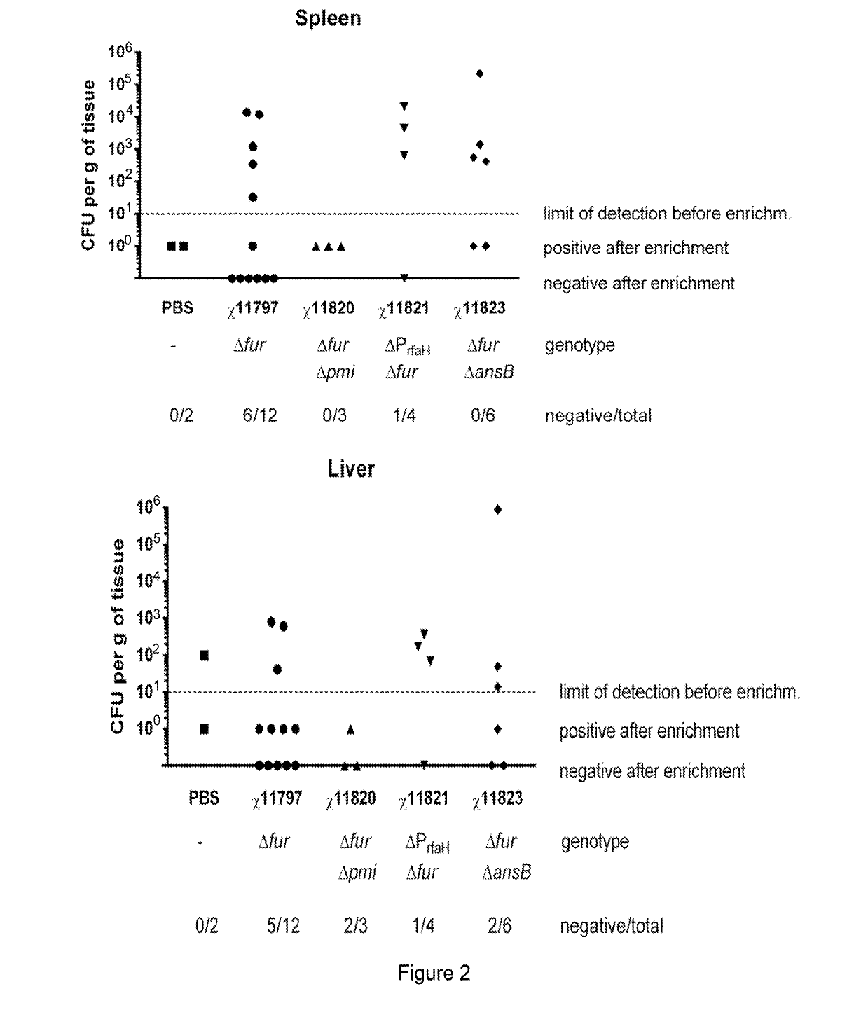

Additionally, in one of the protection studies (Experiment 3 in Table 4), internal organs from all animals were inspected for lesions and bacterial loads after challenge. Birds that died from challenge were necropsied immediately and survivors were euthanized three weeks post challenge and necropsies performed at that time. In Rhode Island Reds that died of fowl typhoid, characteristic lesions included splenomegaly and hepatomegaly and, in some animals, some bronzing of the liver was noted. No other gross lesions were detected in chickens that did not survive the challenge. In contrast spleens and livers in birds vaccinated with χ11797 were, for the most part, not enlarged or congested (Table 4). However, we found nodules in the hearts and observed acute pericarditis in 38% of immunized birds. Furthermore, nineteen days post-challenge, spleen and liver samples were collected from all surviving birds to enumerate S. Gallinarum colonization in each tissue (FIG. 2). In birds vaccinated with χ11797 (Δfur-712), the S. Gallinarum challenge strain was not detectable in 50% of spleen and or 42% of liver samples. The bacterial loads were significantly lower (max. 1.4×104 CFU/g for spleen; 7.9×102 CFU/g for liver) in the remaining, S. Gallinarum positive tissues than in the non-vaccinated birds that succumbed to the infection, where counts were typically around 1×106 CFU per gram of tissue (data not shown).

### Working Example 2

Immunogenicity of S. Gallinarum double mutants. We next examined two distinct genetic strategies for enhancing the immunogenicity of χ11797. It is well established that Salmonella O-antigen is required for efficient colonization of the chicken host. Mutations that result in the gradual loss of O-antigen in vivo can be used in Salmonella vaccine strains to enhance induction of high-antibody titers to outer membrane proteins. Thus, we investigated the possibility that introduction of a Δpmi or an arabinose-regulated rfaH mutation could enhance the immunogenicity of χ11797.

It is likely that all successful pathogens have various means to suppress host immune responses. An example of this in S. Typhimurium is ansB. The product of this gene—L-asparaginase II—suppresses host T cell responses important for clearance of a S. Typhimurium infection and S. Typhimurium ΔansB mutants are attenuated for virulence in mice. Thus, as an alternative approach for enhancing immunogenicity, we examined the effect of ΔansB on virulence and immunogenicity alone or when combined with a Δfur mutation.

We constructed single mutant strains χ11741 (Δpmi-2426) and χ11822 (ΔansB1235) and double mutant strains: χ11798 (Δpmi-2426 Δfur-712), χ11820 (Δfur-712 Δpmi-2426), χ11821 (ΔPrfaH178::TT araC PBAD rfaH Δfur-712) and χ11823 (Δfur-712 ΔansB1235). The absence of detectable Fur was verified in the double mutants (FIG. 1A) and TROMP synthesis was not affected by combining Δpmi-2426, ΔPrfaH178 or ΔansB123 with Δfur-712 (FIG. 1B). Analysis of the LPS profiles of both pmi mutants (χ11741, χ11798) and the ΔPrfaH178 double mutant strains χ11821 indicated that full length O-antigen was produced by both pmi strains and the ΔPrfaH178 mutant only when mannose or arabinose, respectively, was added to the growth medium (data not shown).

We also evaluated acid resistance of the Δpmi mutant strains. Interestingly, strain χ11798 (Δpmi-2426 Δfur-712) was more sensitive to low pH than χ11797 (Δfur-712), even though it was grown in presence of mannose prior to challenge (FIG. 1C). At every time point during challenge, the survival rate of strain χ11798 was significantly less than that of strain χ11797 (P<0.001). It is unlikely that the addition of mannose to strain χ11798 was responsible for the increase in acid sensitivity because strain χ11741 (Δpmi-2426), when grown in LB with mannose, displayed a survival profile identical to wild-type strain 287/91 and we observed no change in survival to acid challenge when mannose was added during growth of χ11797 (Δfur-712) (data not shown).

Since an adequate level of attenuation is critical for designing safe and efficacious vaccines, we examined the virulence of S. Gallinarum strains χ11741 and χ11822, harboring single Δpmi or ΔansB mutations, respectively. The Δpmi mutant χ11741 was partially attenuated, similar to the phenotype observed for S. Typhimurium, while ΔansB mutant χ11822 was fully virulent (Table 3). Strain χ11821 (ΔPrfaH178 Δfur-712), a derivative of hypervirulent strain χ11386 (ΔPrfaH178), was also tested. Introduction of Δfur-712 into χ11386 resulted in complete loss of virulence.

The S. Gallinarum double mutants were then tested for protective efficacy in Rhode Island Reds. Note that while strains χ11820 and χ11798 have the same genotype, the mutations were introduced in different orders, with the Δfur-712 mutation introduced first, before Δpmi-2426, in strain χ11820 and second in strain χ11798. Birds immunized with either χ11820 (Δfur-712 Δpmi-2426) or χ11798 (Δpmi-2426 Δfur-712) were not protected (33% and 22% survival, respectively) (Table 4). A lack of protection was also observed for birds vaccinated with χ11821 (ΔPrfaH178 Δfur-712) (33% survival). Vaccination with χ11823 (Δfur-712 ΔansB1235) resulted in 50% protection, but this result was not significantly different from non-vaccinated controls.

Protective efficacy of S. Gallinarum vaccine strains in brown leghorn chickens. Two vaccine strains: χ11797 (Δfur-712) and χ11798 (Δpmi-2426 Δfur-712) were also tested for protection immunity in Brown Leghorn chickens. In this study, seven-week-old female chickens were vaccinated with a single dose of vaccine by intramuscular (˜2×104 or 2×107 CFU) or oral (˜2×107 CFU) routes and challenged three weeks later with the virulent wild-type vaccine parent strain 287/19. As shown in Table 5, intramuscular immunization with a high dose (2.6×107 CFU) of strain χ11797 provided protection to all vaccinated birds.

TABLE 5
Protective efficacy of S. Gallinarum fur mutants in female Brown Leghorn chickens.

| | | | | | | Heart | | |
|---|---|---|---|---|---|---|---|---|
| | | | Prime | Hepato- | Spleno- | lesions/ | Alive/ | Percent |
| Strain | Genotype | Route | (CFU) | megaly | megaly | pericarditis | total | survival |
| χ11797 | Δfur-712 | IM | 2.6 × 10⁴ | 0/16 (0%)^{a} | 3/16 (19%)^{a} | 2/16 (13%) | 16/16 | 100%^{b} |
| | | IM | 2.6 × 10⁷ | 1/16 (6%)^{a} | 2/16 (12%)^{a} | 3/16 (19%) | 15/16 | 100%^{b} |
| | | Oral | 2.6 × 10⁷ | 14/16 (88%) | 15/16 (94%) | 2/16 (13%) | 8/16 | 50% |
| χ11798 | Δpmi- | IM | 2.2 × 10⁷ | 1/16 (6%)^{a} | 4/16 (25%)^{a} | 5/16 (31%) | 16/16 | 100%^{a} |
| | 2426 | | | | | | | |
| | Δfur-712 | | | | | | | |
| no | — | — | — | 11/15 (73%) | 11/15 (73%) | 5/16 (31%) | 6/16 | 38% |
| vaccine | | | | | | | | |

^{a}Seven-week-old birds were immunized orally or intramuscularly (IM) with the indicated dose of S. Gallinarum vaccine strain.
^{b}P < 0.01 compared to control

Moreover, enlargement of spleen or liver was observed only in 6 and 12% of vaccinated birds post-challenge, respectively. Interestingly, a single low dose (2.6×104 CFU) of χ11797 delivered intramuscularly was also highly protective (100% survival; splenomegaly and hepatomegaly detected in 0 and 19% of birds, respectively). In comparison, only 38% of non-vaccinated birds survived the challenge and spleen and liver lesions were observed in most of them (73%). On the other hand, when delivered orally, strain χ11797 did not protect Brown Leghorns from wild-type challenge in this model. A survival rate of 50% for Brown Leghorns vaccinated with this route was not significantly different than that of non-vaccinated birds. Moreover, the percentage of birds in this group with organ lesions was similar to the control. In contrast, when administrated intramuscularly, strain χ11798 provided significant protection against fowl typhoid (100% survival, P<0.001; lower percentage of birds with lesions relative to control; P<0.01).

### DISCUSSION

In our study we found that deletion of the fur gene in S. Gallinarum resulted in a completely avirulent strain that is highly efficacious as a live vaccine and can protect chickens against fowl typhoid when delivered orally in Rhode Island Red chickens or intramuscularly in Brown Leghorn chickens. These results differ from observations of an S. Typhimurium mutant, which is commonly used as a model for typhoid fever-like infections. While S. Typhimurium Δfur mutants were attenuated when delivered orally or intraperitoneally in mice, they were not found to be substantially immunogenic.

However, the level of attenuation conferred to S. Typhimurium by a fur mutation appears to be strain dependent. When delivered orally, derivatives of S. Typhimurium SL1344 are only attenuated about 10-fold, while S. Typhimurium UK-1 fur mutants are attenuated 1000-fold or more. Differences in rpoS alleles can influence the acid tolerance response of both wild type and fur mutants of Salmonella and may therefore affect other phenotypic aspects of fur mutants.

Alternatively, it is possible that undefined differences between strains may also affect virulence and immunogenicity. In addition, differences in immunogenicity between fur mutants of S. Typhimurium and S. Gallinarum may also be explained by the fact that the disease caused by S. Typhimurium in mice is not exactly the same as that caused by S. Gallinarum in chickens. Support for this view comes from observations that mutations that completely attenuate S. Typhimurium are often insufficiently attenuating for S. Typhi in humans. Since S. Gallinarum and S. Typhi are strictly host-adapted serovars, mechanisms of their pathogenesis are different from the broad-host range S. Typhimurium. Unfortunately, the molecular basis of host specificity as well as the mechanisms determining which type of disease is caused in which animal species are still poorly understood.

Adequate balance between the level of attenuation and immunogenicity is crucial for designing effective live vaccines, but is often difficult to achieve. As we suggest above, the same means of attenuation may result in different levels of attenuation, reactogenicity and/or immunogenicity depending on serovars or strains used for their construction. Protection from disease may also be influenced by the route of administration as well as genetic properties or age of particular breeds such as Rhode Island Red or Brown Leghorn chickens.

Deletions of fur have been introduced into fish pathogens Pseudomonas fluorescens and Edwardsiella ictaluri to generate live attenuated vaccines. A fur mutant of P. fluorescens was attenuated and able to elicit protection in Japanese flounders against P. fluorescens, as well as cross-protection against Aeromonas hydrophila. The authors of that study suggest that the observed cross-protection was related, at least in part, to constitutive production of IROMPs by the P. fluorescens fur mutant. Similarly, arabinose-regulated fur mutants of S. Typhimurium induce antibodies that recognize the IROMPs present in the outer membranes of a number of S. enterica serovars and E. coli. Since our S. Gallinarum fur mutants constitutively synthesize IROMPs (FIG. 1B), it will be interesting to determine how well an S. Gallinarum Δfur mutant such as χ11797 protects chickens against other Salmonella serovars, in particular S. Enteritidis and S. Typhimurium. This will be a topic for future study.

The Δpmi mutant strain χ11741 was moderately attenuated, with an oral LD50 about 2.5 logs higher than its wild-type parent, 287/91 (Table 3). This modest reduction in virulence is similar to the situation seen in S. Typhimurium, where a 3.3-log increase in oral LD50 (for mice) was observed for a pmi mutant grown with mannose. The partial virulence of χ11741 makes this mutant unsuitable for use as a stand-alone vaccine strain. The idea behind combining Δpmi and Δfur in the same strain was that the loss of O-antigen over time would enhance presentation of the IROMPs to the host immune system.

Because it has been argued that the lack of immunogenicity of S. Typhimurium fur mutants is due to an inability to colonize the gut associated lymphoid tissue (GALT), we considered it a plus that the pmi mutant was not fully attenuated and should therefore have a minimal impact on its immunogenicity. We felt that this would reduce the possibility that the double mutant would be over attenuated. However, while the double mutants χ11798 and χ11820 were attenuated, neither strain was protective when administered orally (Table 4). We used a similar strategy by combining fur with the arabinose-regulated rfaH mutation, ΔPrfaH178 mutation, which is not attenuating, and in fact appears to be hypervirulent. Once again, this combination was also not protective (Table 4).

In contrast to our results in Rhode Island Red chicks, S. Gallinarum Δpmi Δfur mutant χ11798 was substantially immunogenic when used to intramuscularly immunize seven-week-old Brown Leghorns (Table 5). Thus, it may be that because the double mutant is more sensitive to low pH than the Δfur strain (FIG. 1C), it does not survive as well during passage through the low pH environment of the proventriculus. If this is the case, pH sensitivity may also help to explain our conflicting results with fur mutant χ11797, which was protective when orally administered to chicks (Table 4), but was less effective when orally administered to older layers (Table 5).

A recent study showed that the proventricular pH in chickens changes during the first few weeks of life, ranging from a pH of about 5 at two days of age to about 3 to 3.5 by fifteen days of age. Thus, it is possible that survival of strain χ11797 was greater in chicks than in the older birds used in our study. When we bypassed the gastric compartment by intramuscular injection, χ11797 was able to elicit a protective response (Table 5). The increased acid sensitivity of χ11798 could account for its lack of immunogenicity in chicks. An alternative interpretation of these results is that because fur, pmi and rfaH all affect outer membrane structure/composition, overexpression of outer membrane proteins (e.g., IROMPs) in the absence of complete O-antigen has a negative influence on immunogenicity, perhaps due to destabilization of outer membrane integrity in vivo. Of course, it is possible that other factors, including possible iron toxicity, could have played a role.

Recently it was shown that S. Typhimurium utilizes a product of ansB gene—L-asparaginase II—to inhibit host T cell responses essential to clearance of Salmonella infection. A canonical function of L-asparaginase II is hydrolyzing L-asparagine to L-aspartate and ammonia. However, beyond the metabolic function, the enzyme plays a role in virulence. Production of L-asparaginase II by Salmonella leads to depletion of exogenous L-asparagine, a metabolite required for T cell proliferation. While an S. Typhimurium ansB mutant was attenuated for virulence in mice, this was not the case for S. Gallinarum in chicks (Table 3) and introduction of a ΔansB mutation into the Δfur mutant strain χ11797 abrogated, rather than enhanced, its immunogenicity (Table 4).

One of the primary goals of our research is to develop a safe and effective orally administered fowl typhoid vaccine for birds. Oral administration of vaccines is, in general, easier to perform than injection and more likely to induce mucosal responses. The results of this study indicate that Δfur mutant χ11797 is safe (Table 3). It is effective in chicks (Table 4), but is not as effective for use as an oral vaccine in older birds (Table 5), though it is substantially immunogenic when delivered by the intramuscular route. It is possible that the problem of efficacy in older birds can be rectified by introduction of a mutation that allows for regulated delayed fur expression, as has been demonstrated to be effective in the S. Typhimurium mouse model. We used a similar strategy to regulate expression of crp in S. Gallinarum with promising results. If this strategy is effective with fur, it may allow us to take advantage of a second mutation in pmi, as we described above.

In conclusion, this study demonstrated that the fur gene is essential for virulence of S. Gallinarum and a fur deletion resulted in complete attenuation of S. Gallinarum. Further, a Δfur mutant is protective against fowl typhoid when used as a live recombinant vaccine following intramuscular administration, or by oral route in young birds.

The claims are not intended to be limited to the embodiments, examples, materials and methods described above.

1. A strain of S. Gallinarum bacteria comprising:
a mutation that prohibits the strain from synthesizing a functional ferric uptake regulator protein, wherein the strain is attenuated and immunogenic in fowl.

2. The strain of claim 1 further comprising a mutation that prohibits the strain from synthesizing phosphomannose isomerase.

3. A strain of S. Gallinarum bacteria comprising:
a mutation that prohibits the strain from synthesizing phosphomannose isomerase, wherein the strain is attenuated and immunogenic in fowl.

4. A vaccine for inoculation against S. Gallinarum infection comprising:
a pharmaceutically acceptable carrier; and
a strain of S. Gallinarum including a mutation that prohibits the strain from synthesizing a functional ferric uptake regulator protein.

5. The vaccine of claim 4 further comprising a mutation that prohibits the strain from synthesizing phosphomannose isomerase.

6. The vaccine of claim 4 wherein the pharmaceutically acceptable carrier is suitable for intramuscular administration or oral administration.

7. A method of making a vaccine comprising:
providing a strain of S. Gallinarum including a mutation that prohibits the strain from synthesizing a functional ferric uptake regulator protein, wherein the strain is attenuated and immunogenic; and
incorporating the strain into a pharmaceutically acceptable carrier.

8. The method of claim 7 further comprising a mutation that prohibits the strain from synthesizing phosphomannose isomerase.

9. The method of claim 7 wherein the pharmaceutically acceptable carrier is suitable for intramuscular administration or oral administration.

10. A method of vaccinating fowl against typhoid, comprising the administration of a strain of S. Gallinarum, which includes a mutation that prohibits the strain from synthesizing a functional ferric uptake regulator protein, in a pharmaceutically acceptable carrier.

11. A method of vaccinating fowl against typhoid, comprising the administration of a strain of S. Gallinarum, which includes a mutation that prohibits the strain from synthesizing phosphomannose isomerase, in a pharmaceutically acceptable carrier.
